(19) United States
(12) Patent Application Publication
(10) Pub. No.: US 2024/0358966 A1
(43) Pub. Date: Oct. 31, 2024

(54) Blood Draw Compatible Open Peripheral Intravenous Catheter System

(71) Applicant: Becton, Dickinson and Company, Franklin Lakes, NJ (US)

(72) Inventors: Srinath B, Tamil Nadu (IN); Karthik MR, Bangalore (IN); Balaji K, Karnataka (IN); Stephen Robert Cassidy, Mahwah, NJ (US); Jonathan Karl Burkholz, Salt Lake City, UT (US); S. Ray Isaacson, Layton, UT (US)

(21) Appl. No.: 18/139,409

(22) Filed: Apr. 26, 2023

(51) Int. Cl. A61M 25/00; A61B 5/15; A61M 25/06; A61M 39/10; A61M 39/22

(52) U.S. Cl. CPC A61M 25/0026; A61B 5/15003; A61B 5/150992; A61M 25/0606; A61M 39/10; A61M 39/22; A61M 2039/229

(57) ABSTRACT

Provided herein is a catheter system including a catheter having a catheter distal end and a catheter proximal end, with the catheter defining a catheter lumen. The catheter system also includes a catheter hub coupled to the catheter proximal end, with the catheter hub further including a hub body having a hub distal end and a hub proximal end, the hub body having a cavity formed therein extending between the hub distal end and the hub proximal end. The catheter hub also includes an end port positioned at the hub proximal end and that provides an opening into the cavity. A split-septum connector of the catheter system is positioned at least partially within the end port, with the split-septum connector configured to seal the cavity when in a closed configuration and provide a fluid path to the catheter lumen when in an open configuration.

### BACKGROUND OF THE INVENTION

### Field of the Invention

Provided herein are devices and systems for use in vascular access, and, in particular, a catheter hub that is directly compatible with a blood draw device to provide for advancement of an instrument through an open peripheral intravenous catheter.

### Description of Related Art

Vascular access devices (VADs) are used in the medical field to access peripheral vasculature of a patient for purposes of infusion therapy and/or blood withdrawal. Common types of VADs include over-the-needle peripheral intravenous catheters (PIVCs), peripherally inserted central catheters (PICCs), central venous catheters (CVCs), and midline catheters. The VAD may be indwelling for short term (days), moderate term (weeks), or long term (months to years). For some applications, an open PIVC catheter is provided where an indwelling catheter in inserted into the vasculature of a patient and extends out therefrom to a catheter hub having a port thereon that provides for coupling of additional instruments or devices thereto.

Instrument delivery devices are often used with VADs to deliver an instrument into the indwelling intravenous (IV) catheter thereof, with the instrument delivery device advancing the instrument beyond the tip of the indwelling catheter. When the instrument delivery device is used to collect blood, as one example, the instrument can be in the form of a flexible tube or catheter, but the instrument may also be a guidewire, obturator, wire, electrical wiring, probe, or sensor(s), in other implementations. Typically, when employing an instrument delivery device with a VAD, such as the open PIVC catheter referenced above, a connector or adapter is included that is positioned between the catheter hub and the instrument delivery device. The adapter includes a coupler at a distal end thereof that connects with a port of the catheter hub and a coupler at a proximal end thereof that connects with the instrument delivery device, with the proximal end coupler often having a needle-free connector therein for receiving the instrument delivery device. When coupled to the catheter adapter, the instrument delivery device may be operated to advance the instrument out therefrom and through a lumen of the catheter adapter, into and then out beyond the tip of the indwelling catheter. The adapter may also include a side port thereon that enables connection of an extension set or fluid delivery system thereto, for introducing fluids into the adapter and to the open PIVC catheter for administering to the patient.

It is recognized that the inclusion of the adapter in the catheter system (between the catheter hub and the instrument delivery device) may increase the difficulty of advancing the instrument into the indwelling catheter. That is, in configurations where a catheter connector is used to connect an instrument delivery device to the catheter hub, a male connection portion of the catheter connector typically couples with the proximal end port of the catheter hub but may not fill a majority of a cavity within the catheter hub. With at least a portion of the cavity in the catheter hub remaining open, a distal end of the male connection portion remains spaced apart from a proximal opening of the lumen defined by the catheter (which is secured to the distal end of the catheter hub) and thus, when attempting to advance the instrument through this cavity (i.e., through the open cavity portion remaining between the male connection portion and the proximal opening of the catheter lumen), the instrument may not remain aligned properly with the catheter lumen. Accordingly, the instrument may become trapped within the cavity of the catheter hub and, as the instrument is further advanced, the instrument may kink and double over on itself, thereby inhibiting advancement of the instrument into and through the catheter.

Accordingly, a need exists in the art for a catheter system that provides for more efficient advancement of an instrument through an open PIVC. The catheter system provides a catheter hub that is directly compatible with a blood draw device, thereby negating the need for a catheter adapter and simplifying advancement of an instrument through the open PIVC.

### SUMMARY OF THE INVENTION

Provided herein is a catheter system that includes a catheter and a catheter hub. The catheter has a catheter distal end and a catheter proximal end and defines a catheter lumen. The catheter hub is coupled to the catheter proximal end and includes a hub body having a hub distal end and a hub proximal end, the hub body having a cavity formed therein extending between the hub distal end and the hub proximal end. The catheter hub also includes an end port provided at the hub proximal end and that provides an opening into the cavity. The catheter system also includes a split-septum connector positioned at least partially within the end port, the split-septum connector configured to seal the cavity when in a closed configuration and provide a fluid path to the catheter lumen when in an open configuration.

In some embodiments, the catheter hub further includes a side port extending out from the hub body between the hub distal end and the hub proximal end, the side port defining a side port lumen in fluid communication with the catheter lumen.

In some embodiments, the side port extends out from the hub body at an angle relative to the catheter lumen of between 15-165 degrees.

In some embodiments, the side port is a female luer connection.

In some embodiments, the catheter system further includes a needle-free access connector coupled to the side port.

In some embodiments, the cavity is a cylindrical cavity having a constant diameter along at least a majority of a length thereof between the hub distal end and the hub proximal end.

In some embodiments, the catheter system further includes a blood draw device, the blood draw device coupled to the end port and/or the split-septum connector.

In some embodiments, the blood draw device includes a secondary catheter, an introducer having a proximal end and a distal end and a top surface and a bottom surface that define an inner volume configured to movably receive the secondary catheter, an actuator movably coupled to the introducer and configured to move relative to the introducer to move a distal end of the secondary catheter from a position within the introducer to a position outside the introducer housing and past the catheter distal end of the catheter, and a connector positioned at the distal end of the introducer that is coupled to the end port and/or the split-septum connector, the connector comprising a blunt cannula and a pair of locking arms.

In some embodiments, the split-septum connector includes a first ring and a second ring spaced apart laterally from the first ring, wherein the pair of locking arms engages the split-septum connector by snapping-in between the first ring and the second ring.

In some embodiments, when the blood draw device is coupled to the end port and/or the split-septum connector, the blunt cannula penetrates through the split-septum connector and into the cavity, with each of the blunt cannula and the cavity having a length such that the blunt cannula extends approximately to a distal end of the cavity.

In some embodiments, extension of the blunt cannula to approximately the distal end of the cavity places a lumen of the blunt cannula adjacent the catheter lumen.

In some embodiments, the catheter hub further includes a wedge positioned within the cavity adjacent the hub distal end, the wedge coupled to the catheter proximal end to retain the catheter to the catheter hub, and wherein the wedge includes a wedge lumen formed therein, and extension of the blunt cannula to approximately the distal end of the cavity directly aligns the lumen of the blunt cannula with the wedge lumen.

In some embodiments, the split-septum connector is positioned entirely within the end port, and wherein the pair of locking arms engages a groove formed on an outer surface of the end port.

In some embodiments, the catheter system further includes a needle introducer coupled to the split-septum connector, with a needle extending through the catheter hub and through the catheter lumen, and wherein the split-septum connector is configured to have an initial configuration where a first portion of the split-septum connector is separated from a second portion of the split-septum connector, such that the needle passes through the split-septum connector without making contact therewith.

In some embodiments, the catheter hub comprises an open peripheral intravenous catheter (PIVC) catheter hub.

In some embodiments, the split-septum connector includes a side port formed thereon, with an integrated extension tube extending out from the side port.

Also provided herein is a catheter system that includes a catheter and a catheter hub. The catheter has a catheter distal end and a catheter proximal end and defines a catheter lumen. The catheter hub is coupled to the catheter proximal end and includes a hub body having a hub distal end and a hub proximal end, the hub body having a cavity formed therein extending between the hub distal end and the hub proximal end. The catheter hub also includes an end port provided at the hub proximal end and that provides an opening into the cavity. The catheter system also includes a fluid cut-off positioned at or adjacent the end port, the fluid cut-off configured to seal the cavity when in a closed configuration and provide a fluid path to the catheter lumen when in an open configuration.

In some embodiments, the fluid cut-off includes a stop cock valve within the cavity adjacent the end port and an actuator positioned on an outer surface of the hub body and operable to open and close the stop cock valve.

In some embodiments, the catheter system further includes a split-septum connector within the cavity and proximal from the stop cock valve.

In some embodiments, the catheter system further includes a deformable tubing coupled to the end port and extending our proximally therefrom, with the fluid cut-off being a slide clamp positioned on the deformable tubing and actuatable between an open position and a closed position to selectively allow fluid flow through the deformable tubing.

In some embodiments, the catheter system further includes a blood draw device coupleable to the proximal coupler of the connector, with the blood draw device configured to advance a secondary catheter past the catheter distal end of the catheter and including a connector that is coupled to the end port. The connector further includes a blunt cannula and a pair of locking arms and, when the blood draw device is coupled to the end port and when the fluid cut-off is in the open configuration, the blunt cannula penetrates into the cavity, with the blunt cannula and the cavity each having a length such that the blunt cannula extends approximately to a distal end of the cavity.

### BRIEF DESCRIPTION OF THE DRAWINGS

FIG. 1 is a perspective view of a catheter system, including an open PIVC catheter assembly and attached blood draw device and fluid delivery system, according to an aspect or embodiment of the present application;

FIG. 2 is a top view of a portion of the catheter system of FIG. 1;

FIG. 3 is a cross-sectional view of the catheter system of FIG. 1;

FIG. 4 is a cross-sectional view of the catheter system of FIG. 1, showing an instrument in a first position;

FIG. 5 is a cross-sectional view of the catheter system of FIG. 1, showing an instrument in a second position;

FIG. 6 is a perspective view of a catheter system, including an open PIVC catheter assembly and attached needle introducer, according to an aspect or embodiment of the present application;

FIG. 7A illustrates the catheter system of FIG. 6 with a needle inserted through a catheter hub and separable split-septum connector, according to an aspect or embodiment of the present application;

FIG. 7B illustrates the catheter system of FIG. 6 with a needle retracted from the catheter hub and separable split-septum connector;

FIG. 8 illustrates the catheter system of FIG. 6 with a split-septum connector removed from the catheter hub when the needle introducer is attached;

FIG. 9A is a perspective view of an open PIVC catheter assembly with an integrated stop cock mechanism in an open configuration, according to an aspect or embodiment of the present application;

FIG. 9B illustrates the open PIVC catheter assembly of FIG. 9A with the integrated stop cock mechanism in a closed configuration;

FIG. 10A is a perspective view of an open PIVC catheter assembly with attached tubing and a clamp in an open configuration, according to an aspect or embodiment of the present application;

FIG. 10B illustrates the open PIVC catheter assembly of FIG. 10A with the clamp in a closed configuration;

FIG. 11 is a perspective view of a catheter system, including an open PIVC catheter assembly and attached blood draw device, according to another aspect or embodiment of the present application;

FIG. 12 is a cross-sectional view of the catheter system of FIG. 11; and

FIG. 13 is a perspective view of a catheter system, including an open PIVC catheter assembly and attached blood draw device, according to another aspect or embodiment of the present application.

### DESCRIPTION OF THE INVENTION

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a catheter insertion device in preparation for or during use. Similarly, in this application, the terms “in the distal direction” and “distally” mean in the direction toward the distal tip of the needle or catheter of the system, and the terms “in the proximal direction” and “proximally” mean in the direction opposite the direction of the distal tip of the needle or catheter.

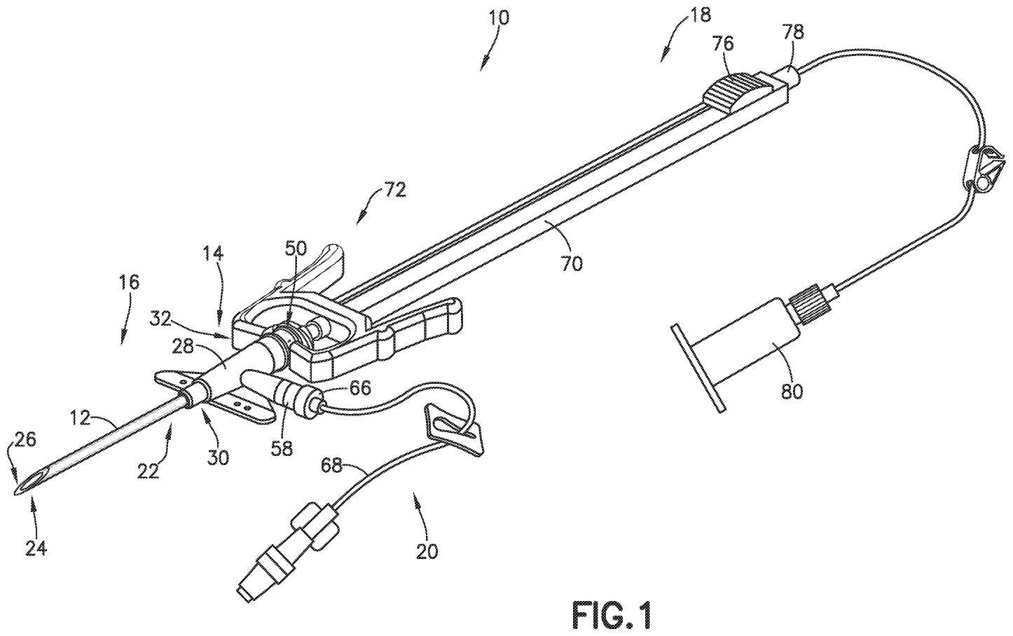

Referring to FIG. 1, a catheter system 10 is shown, according to an aspect or embodiment of the disclosure. The catheter system 10 includes a catheter 12 and associated catheter hub 14 that may collectively provide an open PIVC catheter assembly 16. The catheter system 10 further includes an instrument delivery device 18 and/or additional extension set or fluid delivery system 20 that may be connected directly to the catheter hub, to provide for delivery of an instrument and/or fluid into and through the catheter 12.

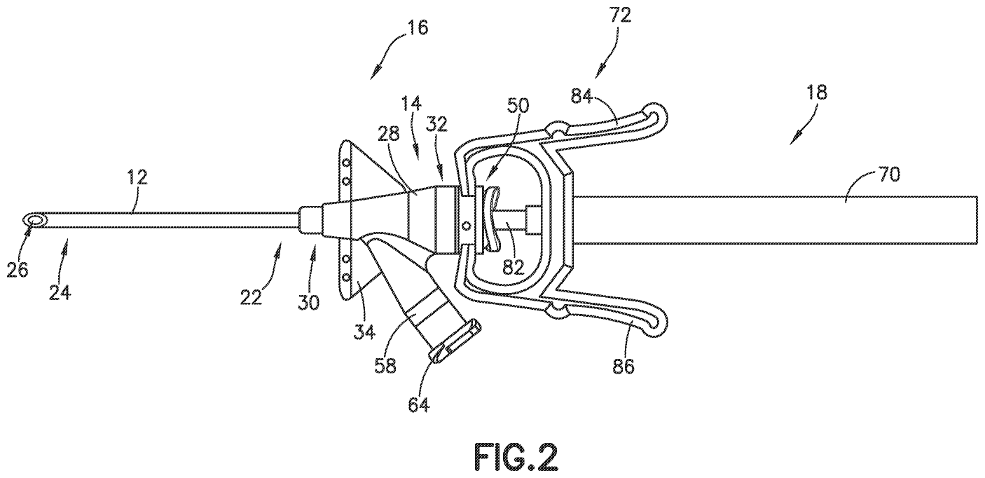

As shown in FIGS. 1-5, the open PIVC catheter assembly 16 includes a catheter 12 and catheter hub 14 that provide intravenous access to the vasculature of a patient. In some embodiments, the catheter 12 may include a peripheral intravenous catheter, but it is recognized that catheter 12 could also be provided as a midline catheter, or a peripherally-inserted central catheter. Catheter 12 may be formed of any suitable material and may be of any useful length, as known to those of skill in the art. A proximal end 22 of the catheter 12 is coupled to catheter hub 14 and extends distally away therefrom to a distal end 24 of the catheter 12. The catheter 12 defines a lumen 26 therein via which fluids may be administered to a patient, an instrument may be introduced into the vasculature of the patient, or blood may be drawn from the patient.

The catheter hub 14 is constructed of a main hub body 28 having a distal end 30 and a proximal end 32, along with stabilizing wings 34 extending out from the main hub body 28 that provide for securing of the hub to the skin of a patient. The main hub body 28 defines a cavity 36 therein that extends between the distal end 30 and the proximal end 32 of the catheter hub 14. According to aspects or embodiments of the disclosure, the cavity 36 is configured as a generally cylindrically shaped cavity that has a constant diameter along at least a majority of a length 37 of the cavity 36. A hub end port 38 is provided at the proximal end 32 of the main hub body 28 that provides access into the cavity 36. An opening 40 is also formed at the distal end 30 of the main hub body 28 that extends into the cavity 36, with the opening 40 also receiving the proximal end 22 of the catheter 12 therein. In some embodiments, the opening 40 is sized to have a similar diameter to catheter 12 so that catheter 12 is secured within the opening, thereby anchoring the catheter 12 to the catheter hub 14.

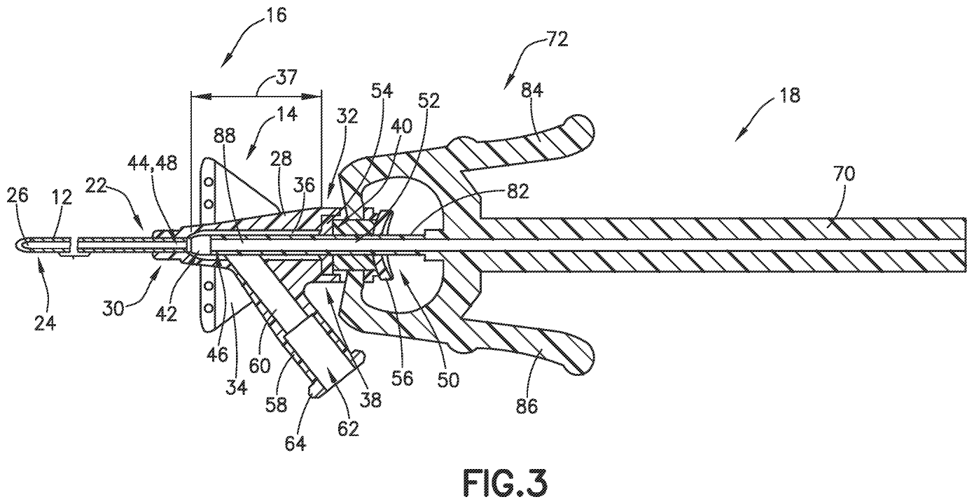

As shown in FIG. 3, according to some aspects or embodiments, the catheter hub 14 may include a wedge 42 positioned within the cavity 36 of main hub body 28. The wedge 42 is positioned within the cavity 36 adjacent distal end 30 of the catheter hub 14 such that the wedge 42 may be seated within the cavity 36. The wedge 42 includes openings 44, 46 formed therein at opposing distal and proximal ends thereof, with a wedge lumen 48 extending between the distal and proximal openings 44, 46. The wedge 42 is positioned within cavity 36 so as to be aligned with the distal opening 40 in the main hub body 28, with the distal opening 44 in wedge 42 aligned with the distal opening 40 in the main hub body 28. With the openings of the wedge 42 and main hub body 28 aligned, the catheter 12 extends through the distal opening 40 in the main hub body 28 and into the distal opening 44 in wedge 42—with the distal opening 44 in wedge 42 sized such that the catheter 12 is secured within the opening 44 and secured to the wedge 42, thereby anchoring the catheter 12 to the catheter hub 14.

In accordance with some aspects and embodiments, the hub end port 38 at the proximal end 32 of main hub body 28 is configured as a receptacle that receives a proximal coupler 50 therein. The proximal coupler 50 may be configured as a split-septum, needle-free connector (NFC) such as, e.g., Q-Syte™ or SmartSite™ NFCs from Becton, Dickinson and Co., or any other appropriate split-septum NFC, that is fitted partially within the proximal hub end port 38—hereafter “split-septum connector 50.” The split-septum connector 50 includes a split septum 52 therein that serves as an access control feature whereby a peripheral device (e.g., a blood draw device or a vascular access probe for in-vein digital measurement of patient data such as temperature, pH, lactate, and/or other blood-based measurements) can be physically and fluidically coupled to the catheter hub 14, such that the split-septum connector 50 allows the catheter hub 14 to be accessed multiple times by the peripheral device. In some embodiments, where the peripheral device is a blood draw device that introduces a secondary catheter into catheter 12, the split-septum connector 50 functions as a blood control feature that may be accessed multiple times by the blood draw device to draw the blood from a vein of the patient. The split-septum connector 50 also includes a pair of connection rings 54, 56 formed about a perimeter thereof, with a first ring 54 and a second ring 56 spaced laterally apart. The connection rings 54, 56 provide for engagement of the instrument delivery device 18 to the catheter hub 14, as will be explained in further detail below.

In accordance with some aspects and embodiments, the catheter hub 14 also includes and/or defines one or more additional ports, such as a side port 58 that extends out from the main hub body 28. The side port 58 may be formed in a desired position along a length of the main hub body 28, between the distal end 30 and the proximal end 32, to facilitate flushing and/or fluid transfer via the side port 58. The side port 58 defines a lumen 60 that is in fluid communication with the cavity 36, so as to provide access to and be in fluid communication with the catheter lumen 26. In some embodiments, the side port 58 is configured as a female luer connection that includes a tapered inner cavity 62 and a threaded outer surface 64, with the female luer connection configured to mate with a corresponding male luer connection of a connector 66 (FIG. 1) that is coupled to the side port 58. In some embodiments, the connector 66 may be a NFC that provides controlled access to the catheter hub 14, with the extension set or fluid delivery system 20 connected to the side port 58 through the NFC 66. The NFC 66 may be configured as a split-septum NFC such as, e.g., Q-Syte™ or SmartSite™ NFCs from Becton, Dickinson and Co., or any other appropriate split-septum NFC. Via connector 66, the extension set or fluid delivery system 20 may be connected to side port 58 so as to be in fluid communication with the lumen 60 of the side port 58 and may deliver fluid, remove fluid, flush fluid, and/or the like, such as providing fluid from an attached IV bag (not shown), as one example. In such embodiments, side port 58 can enable flushing of the split-septum connector 50 and/or a space within cavity 36 between the side port 58 and the split-septum connector 50.

While not shown in FIG. 1, it is recognized that some embodiments of catheter hub 14 may have the extension set or fluid delivery system 20 integrated therewith via side port 58. That is, extension set or fluid delivery system 20 (i.e., extension tubing 68 thereof) may be integrated directly with side port 58 rather than side port 58 being provided as an open female luer connection to which a separate connector 66 would be attached (with extension tubing 68 connected thereto).

In some embodiments, the arrangement of side port 58 can be such that the catheter hub 14 is constructed as, for example, a Y-shaped or a T-shaped catheter hub. In non-limiting embodiments, side port 58 extends from main hub body 28 at an angle relative to the main hub body 28 that is not 72 degrees (e.g., side port 58 extends at an angle of, for example and without limitation, 15-165 degrees, with all values and subranges therebetween inclusive). In non-limiting embodiments, more than one side port 58 is included in catheter hub 14.

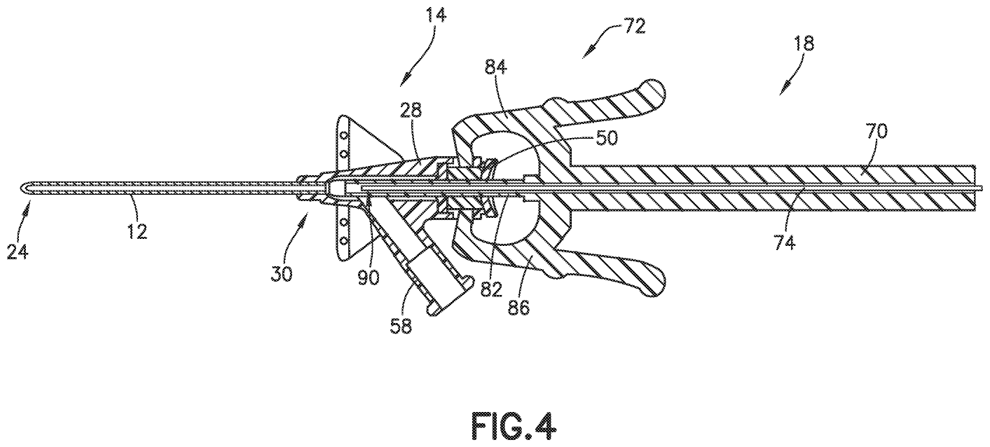

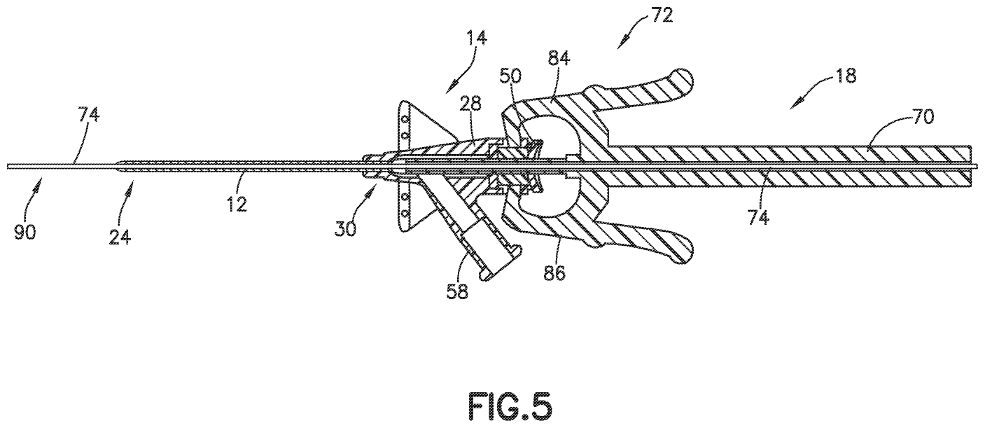

As shown in FIGS. 1-5, the instrument delivery device 18 of catheter system 10 is coupled to the split-septum connector 50 provided in the hub end port 38 of catheter hub 14. According to some aspects or embodiments, the instrument delivery device 18 includes an introducer 70, a connector 72, an instrument 74 (FIGS. 4 and 5), and an actuator 76. The instrument 74 may be any of a number of devices suitable for insertion into the vasculature of the patient, including a secondary catheter, guidewire, or probe, as examples. A portion of the actuator 76 is configured to be advanced along a top surface of the introducer 70, which in turn, facilitates the advancing of the instrument 74 through the introducer 70, the connector 72, the catheter hub 14, and distally out through and past the catheter 12. In some embodiments, a luer adapter, luer lock access device (LLAD), or other connecting device 78 may be provided at a proximal end of the introducer 70 to enable connection of another component 80 to the instrument delivery device 18, such as a syringe or vacutainer to facilitate a blood draw. According to one embodiment, the instrument delivery device 18 acts as a blood draw device that inserts a secondary catheter through the catheter 12 to facilitate a blood draw from the patient, with blood collected in the syringe or vacutainer attached to the device, and thus reference hereafter is made to a blood draw device 18 and secondary catheter 74.

As shown in more detail in FIGS. 3 and 4, the connector 72 of the blood draw device 18 is configured to be physically and fluidically coupled to the introducer 70 and configured to couple the introducer 70 to the catheter hub 14. The connector 72 has a blunt cannula 82, a first arm 84, and a second arm 86, with the connector 72 also defining a lumen 88 extending through the blunt cannula 82. The blunt cannula 82 is disposed between the first arm 84 and the second arm 86 and may have an inner diameter (a diameter of a surface at least partially defining the lumen 88) that is similar to or slightly larger than an outer diameter of a portion of the secondary catheter 74, such that the lumen 88 of the connector 72 can receive a portion of the secondary catheter 74 when the secondary catheter is advanced therethrough. For securing the connector 72 to the catheter hub 14, the first arm 84 and the second arm 86 act to grip the split-septum connector 50 positioned in the hub end port, with the first and second arms engaging the split-septum connector by snapping-in between the first ring and the second ring thereof.

To provide a desired engagement between the blood draw device 18 and the catheter hub 14, the catheter hub 14 is specifically configured and sized to receive the blunt cannula 82 of connector 72, as best shown in FIG. 3. That is, the catheter hub 14 is constructed such that the cavity 36 provided in the main hub body 28 is configured to receive the blunt cannula 82 in a manner that provides for an accurate and reliable advancement of the secondary catheter 74 into the lumen 26 of catheter 12. As previously described, the cavity 36 has a cylindrical profile with a constant diameter along at least a majority of the length 37 of the cavity 36, such that the blunt cannula 82 may be received and accurately guided into the cavity 36 without obstruction when the connector 72 is engaged with the catheter hub 14 (via split-septum connector 50). Furthermore, the cavity 36 is configured to have a length 37 designed to accommodate the full length of the blunt cannula 82, such that when the blood draw device 18 is coupled to the catheter hub 14 (via split-septum connector 50), the blunt cannula 82 extends entirely through the cavity 36 to a location where the lumen 88 of the connector 72 is directly adjacent the lumen 48 of wedge 42 (or adjacent the lumen 26 of catheter 12 if no wedge 42 is included). With the blunt cannula 82 positioned adjacent the wedge 42 in this manner, a distal opening of the lumen 88—which runs through the blunt cannula 82—is brought directly adjacent the wedge lumen 48 and into proximity with the proximal end 22 of the catheter 12, such that the lumen 88 in blunt cannula 82 is directly aligned with the wedge lumen 48.

As described above, at least a portion of the secondary catheter 74 is movably disposed within the introducer 70. A proximal end portion of the secondary catheter 74 is coupled to the actuator 76 and, in this manner, the actuator 76 can be moved relative to the introducer 70 to move the secondary catheter 74 between a first position, in which the secondary catheter 74 is disposed within the introducer 70 (e.g., the entire secondary catheter 74 is disposed within the introducer 70 or within the introducer 70 and the connector 72) and a second position, in which the distal end portion 90 of the secondary catheter 74 is at least partially disposed in a position distal to the connector 72 and the catheter hub 14 when the connector 72 is coupled to the catheter hub 14.

Referring now to FIGS. 4 and 5, changing of the blood draw device 18 between a first configuration and second configuration is shown. The blood draw device 18 can be in the first configuration prior to use and can be transitioned by a user (e.g., a doctor, physician, nurse, technician, phlebotomist, and/or the like) from the first configuration (FIG. 4) to the second configuration (FIG. 5) to dispose at least the distal end portion 90 of the secondary catheter 74 in a distal position relative to the introducer 70 (e.g., within or distal to the indwelling catheter 12).

The blood draw device 18 is shown in the first configuration of FIG. 4 when the secondary catheter 74 is disposed in the first position within the introducer 70. In some embodiments, substantially the entire secondary catheter 74 is disposed within the introducer 70 when the secondary catheter 74 is in the first position. In other embodiments, the secondary catheter 74 is disposed within the introducer 70 and the connector 72 when secondary catheter 74 is in the first position.

The actuator 76 (FIG. 1) is disposed in a proximal position when the blood draw device 18 is in the first configuration, and the user may engage the actuator 76 to move it relative to the introducer 70, which in turn, moves the secondary catheter 74 from the first position (e.g., disposed within the introducer 70) toward the second position. In this manner, the secondary catheter 74 is moved through the inner volume of the introducer 70 and through the lumen 88 of the connector 72.

The blood draw device 18 is in the second configuration of FIG. 5 when the secondary catheter 74 is disposed in the second position. The second position of the secondary catheter 74 is reached when the distal end portion 90 of the secondary catheter 74 is placed in a desired position relative to a distal end 30 of the catheter hub 14 and into/through the catheter 12. In some instances, for example, a distal end 90 of the secondary catheter 74 can be substantially flush with a distal end 24 of the catheter 12 when the secondary catheter 74 is in the second position. In other instances, the distal end 90 of the secondary catheter 74 can extend a predetermined distance beyond the distal end 24 of the catheter 12 (e.g., distal to the distal end 24 of the catheter 12), such that the distal end 90 of the secondary catheter 74 is positioned within the vein at a predetermined distance beyond the distal end 24 of the catheter 12 (e.g., a position within a vein that is substantially free from debris (e.g., fibrin/blood clots) otherwise surrounding the distal end 24 of the catheter 12).

With the secondary catheter 74 in the second position (e.g., with the blood draw device 18 in the second configuration shown, for example, in FIG. 5), the user can establish fluid communication between a component 80 (e.g., fluid reservoir, fluid source, syringe, and/or the like) and the secondary catheter 74. With the secondary catheter 74 in fluid communication with the component 80, the blood draw device 18 can then aspirate a volume of blood from the vein based, at least in part, on disposing the distal end 90 of the secondary catheter 74 at the predetermined and/or desired distance beyond the distal end 24 of the indwelling catheter 12.

Beneficially, the construction of catheter hub 14 and the mating thereof with blood draw device 18 facilitates advancement of the secondary catheter 74 from the first position to the second position. That is, with the cavity 36 of catheter hub 14 having a length 37 such that it accommodates a full length of the blunt cannula 82, the blunt cannula 82 may be positioned adjacent the proximal end 22 of catheter 12 (or the wedge 42), such that the lumen 88 is brought directly adjacent to and into alignment with the lumen 26 of catheter 12. Accordingly, the secondary catheter 74 can be advanced through the lumen 88 of connector 72 and into the lumen 26 of catheter 12 in a straight line-without any opportunity for the secondary catheter 74 to deflect or otherwise become trapped/caught within the cavity 36 of the catheter hub 14.

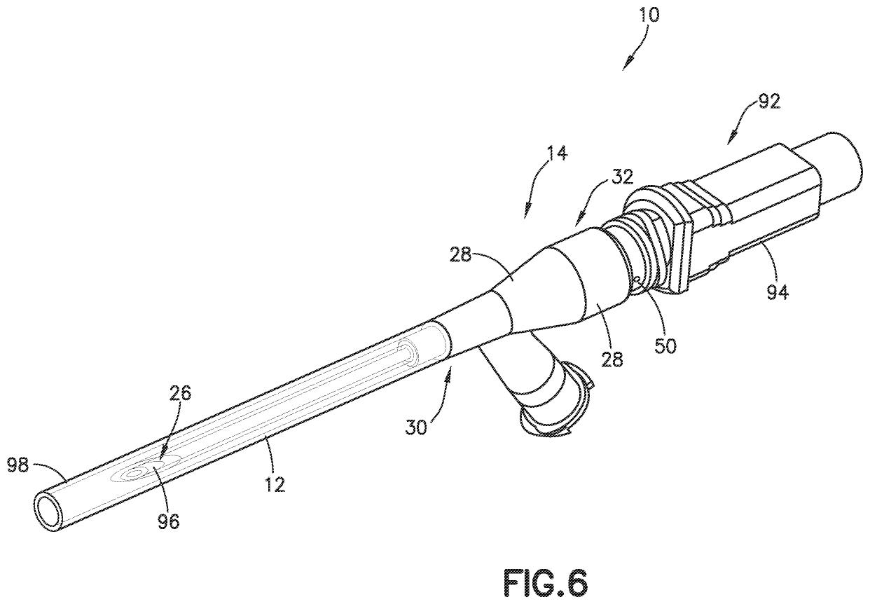

Referring now to FIG. 6, the open PIVC catheter assembly 16 (including catheter 12 and associated catheter hub 14) described above is shown included in a catheter system 10 in accordance with another aspect or embodiment of the disclosure. In the usage of catheter system 10 shown in FIG. 6, a needle introducer 92 is provided for use with the open PIVC catheter assembly 16 to provide for initial insertion of the catheter 12 into the vascular of the patient.

As shown in FIG. 6, the needle introducer 92 generally includes a needle hub 94 and a needle 96. The needle hub 94 is configured to be grasped by a user during insertion of the needle 96 and catheter 12 into the vasculature of the patient. The needle hub 94 is positioned at the proximal end 32 of the catheter hub 14 and is secured thereto via connection to the split-septum connector 50. The needle 96 extends out distally from the needle hub 94, with the needle 96 passing through the split-septum connector 50 and extending through the catheter hub 14 and into/through the lumen 26 of catheter 12. A needle cover 98 may be coupled to the distal end 32 of the catheter hub 14 and positioned over the catheter 12 and needle 96, so as to prevent incidental contact with the tip of needle 96.

In operation of the needle introducer 92, the needle cover 98 may first be removed and a user may then grasp the needle hub 94 and apply a pushing force thereto that causes the needle 96 to pierce the skin of a patient, with a further pushing force then causing the needle 96 and the catheter 12 to be inserted into the vasculature of the patient. Upon insertion of the needle 96 and catheter 12 into the patient, the user may then pull back on the needle hub 94 to retract the needle 96 out from the catheter 12, with the needle hub 94 being disconnected/separated from the split-septum connector 50 and the needle 96 pulled out from the catheter hub 14 through the split-septum connector 50.

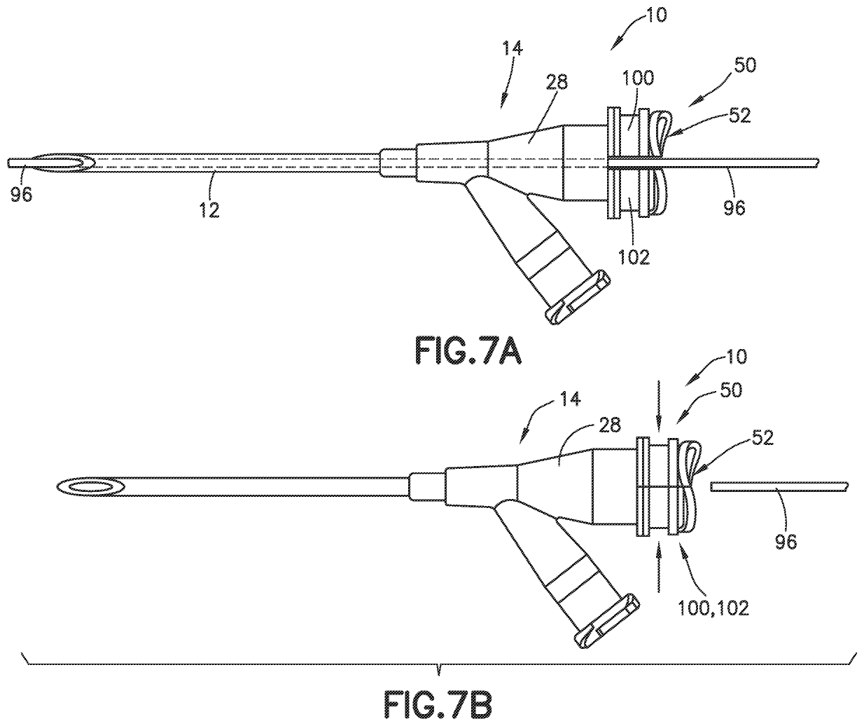

Referring now to FIGS. 7A and 7B, in some embodiments, the split-septum connector 50 may be specifically configured to accommodate passage of the needle 96 therethrough without comprising the integrity of the split septum 52. That is, as the needle 96 is positioned to pass through the split septum 52 of connector 50 for a prolonged period of time when the catheter system 10 is provided in its in-the-package configuration (i.e., prior to actual use of the needle introducer 92), the split-septum connector 50 may be configured to prevent deformation (i.e., compression setting) thereof during such an in-the-package configuration. As shown in the embodiment of FIGS. 7A and 7B, in some aspects or embodiments, the split-septum connector 50 may be positionable in an initial configuration (FIG. 7A) where a first portion 100 of the split-septum connector 50 is separated from a second portion 102 of the split-septum connector 50. In this initial configuration, the needle 96 passes through the split-septum connector 50 without making contact therewith, so as to prevent deformation of the split-septum connector 50 when the catheter system 10 is in its in-the-package configuration.

As shown in FIG. 7B, the first and second portions 100, 102 of the split-septum connector 50 may be joined subsequent retraction of the needle 96 from the connector 50. That is, insertion the needle 96/catheter 12 into the vasculature of the patient, and upon a subsequent removal/retraction of the needle 96 from the split-septum connector 50 and catheter hub 14, the split-septum connector 50 may be pressed from the sides thereof to close or join the first and second portions 100, 102 and snap the split-septum connector 50 locked. Upon the split-septum connector 50 being closed in this manner, the split-septum connector 50 may function in a normal manner-sealing off the cavity 36 within catheter hub 14 until introduction of a device/component through the split septum 52.

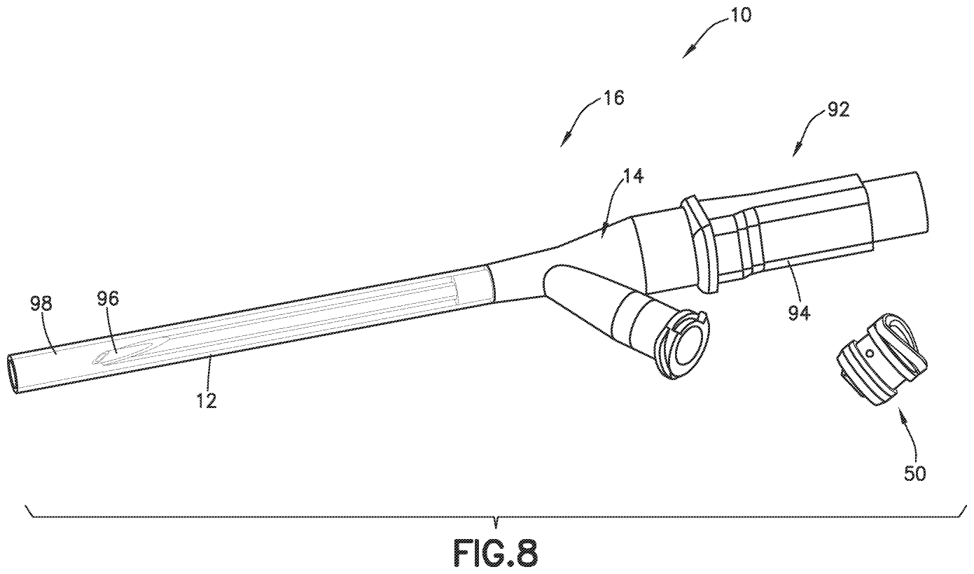

In still other embodiments, and as shown in FIG. 8, in order to prevent compression setting or other deformation of the split-septum connector 50 while the catheter system 10 is in its in-the-package configuration, the split-septum connector 50 may be provided separate from the catheter hub 14 while the catheter system 10 is in its in-the-package configuration. In this configuration, the split-septum connector 50 may be joined to the catheter hub 14 subsequent to use of the needle introducer 92 to insert the needle 96/catheter 12 into the vasculature of the patient, and after disconnection and removal of the needle introducer 92 from the catheter hub 14.

According to additional aspects or embodiments of the disclosure, the open PIVC catheter assembly 16 (including catheter 12 and associated catheter hub 14) described above may have other devices connected thereto or included therein that provide a fluid cut-off to seal the cavity 36 of catheter hub 14 from the external environment. The fluid cut-off may address the issue described above regarding compression setting or other deformation occurring in a split-septum connector 50 during storage of the catheter system 10 in its in-the-package configuration, when a needle introducer 92 is connected to the open PIVC catheter assembly 16. The fluid cut-off may be provided in place of or in addition to a split-septum connector 50, according to various embodiments, with the fluid cut-off accommodating passage of the needle 96 therethrough during storage of the catheter system 10 in its in-the-package configuration.

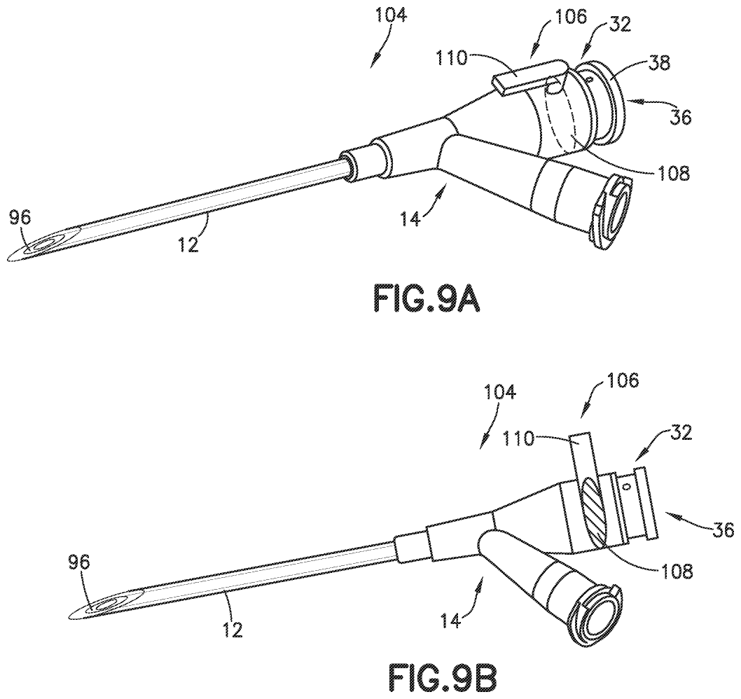

Referring now to FIGS. 9A and 9B, an open PIVC catheter assembly 104 is shown as including a fluid cut-off in the form of a stop cock mechanism 106 incorporated into the catheter hub 14, in accordance with another aspect or embodiment of the disclosure. The stop cock mechanism 106 includes a stop cock valve 108 positioned within the cavity 36 of catheter hub 14, adjacent the hub end port 38, and an actuator 110 positioned on an outer surface of the hub main body. The actuator 110 may be configured as a lever or switch that is operable/movable between a first and second position to open and close the stop cock valve 108, thereby providing an opening into cavity 36 or scaling off cavity 36, respectively.

When a needle introducer 92 is coupled to the catheter hub 14 (as shown in FIG. 6, for example) and stored in its in-the-package configuration (i.e., prior to actual use of the needle introducer 92), the stop cock valve 108 is maintained in its open configuration as shown in FIG. 9A, so as to allow the needle 96 to pass through the stop cock valve 108 and on through the cavity 36 of catheter hub 14 and catheter 12. Upon use of the needle introducer 92 to insert the needle 96/catheter 12 into the vasculature of the patient, and upon disconnection and removal of the needle introducer 92 from the catheter hub 14—with the needle 96 withdrawn from the catheter hub 14 and out through stop cock valve 108—the stop cock valve 108 may be closed (via moving of actuator 110) to seal off the cavity 36 within catheter hub 14, as shown in FIG. 9B. Upon closing of the stop cock valve 108, devices or components may be coupled to the proximal end 32 of catheter hub 14, such as a blood draw device 18 as described in detail above or another instrument delivery device. Subsequently, when it is desired to introduce an instrument (e.g., secondary catheter 74) into the catheter hub 14 and out through catheter 12, the stop cock valve 108 may be opened to enable advancement of the instrument into the catheter 12.

In some embodiments, upon use and removal of the needle introducer 92 from the catheter hub 14, a split-septum connector 50 may be provided in the hub end port 38. The split-septum connector 50 is positioned in the hub end port 38 proximal from the stop cock valve 108, with the split-septum connector 50 functioning to control access into the catheter hub 14 and catheter 12. As described in detail above, an instrument delivery device such as blood draw device 18 may be connected to the split-septum connector 50 and penetrate through the septum 52 thereof (via blunt cannula 82) to advance a secondary catheter 74 into the catheter 12.

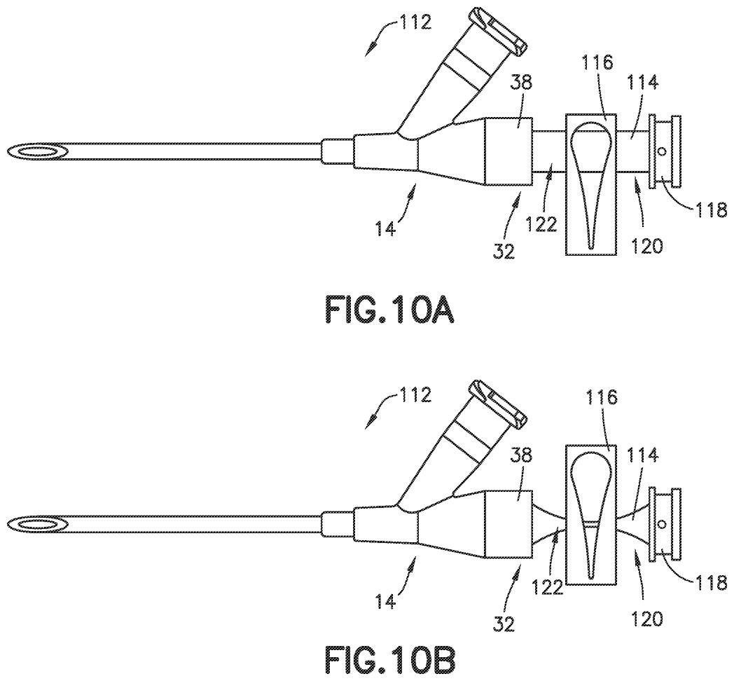

Referring now to FIGS. 10A and 10B, an open PIVC catheter assembly 112 is shown in accordance with another aspect or embodiment of the disclosure, with the open PIVC catheter assembly 112 including deformable tubing 114 and a fluid cut-off in the form of a slide clamp 116 at the proximal end 32 of the catheter hub 14. The deformable tubing 114 is connected to hub end port 38 and extends out proximally therefrom, with a connector 118 provided at a proximal end 120 of the deformable tubing 114 in one embodiment. The deformable tubing 114 may be comprised of silicone or another suitable material that may be deformed, such as being pinched by slide clamp 116. The slide clamp 116 is positioned along a length of the tubing 114 between the hub end port 38 and the connector 118 and is operable in an open configuration and a closed configuration to selectively close off an interior lumen 122 of the tubing 114.

When a needle introducer 92 is coupled to the connector 118 on the proximal end 120 of deformable tubing 114 and the catheter system 10 is stored in its in-the-package configuration (i.e., prior to actual use of the needle introducer 92), the slide clamp 116 is maintained in its open configuration, as shown in FIG. 10A, so as to provide an open lumen 122 within tubing 114 that allows the needle 96 to pass through the tubing 114 and on through the cavity 36 of catheter hub 14 and catheter 12. Upon use of the needle introducer 92 to insert the needle 96/catheter 12 into the vasculature of the patient, and upon disconnection and removal of the needle introducer 92 from the connector 118—with the needle 96 withdrawn from the catheter hub 14 and out through tubing 114—the slide clamp 116 may be closed to clamp shut deformable tubing 114 and seal off the cavity 36 within catheter hub 14 as shown in FIG. 10B. Upon closing of the slide clamp 116, devices or components may be coupled to the connector 118 at proximal end of tubing 114, such as a blood draw device 18 as described in detail above or another instrument delivery device. Subsequently, when it is desired to introduce an instrument (e.g., secondary catheter 74) into the catheter hub 14 and out through catheter 12, the slide clamp 116 may be opened to enable advancement of the instrument through lumen 122 of tubing 114 and into the catheter 12.

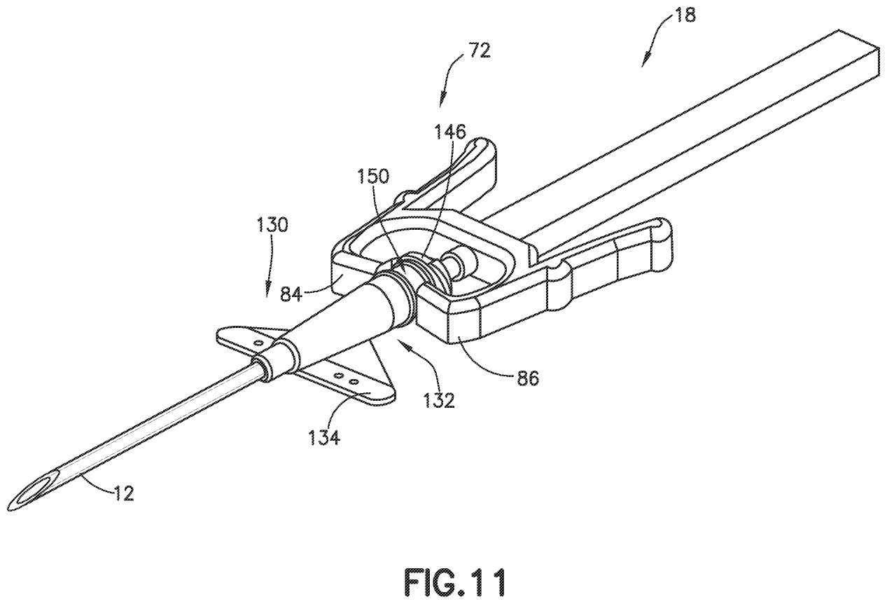

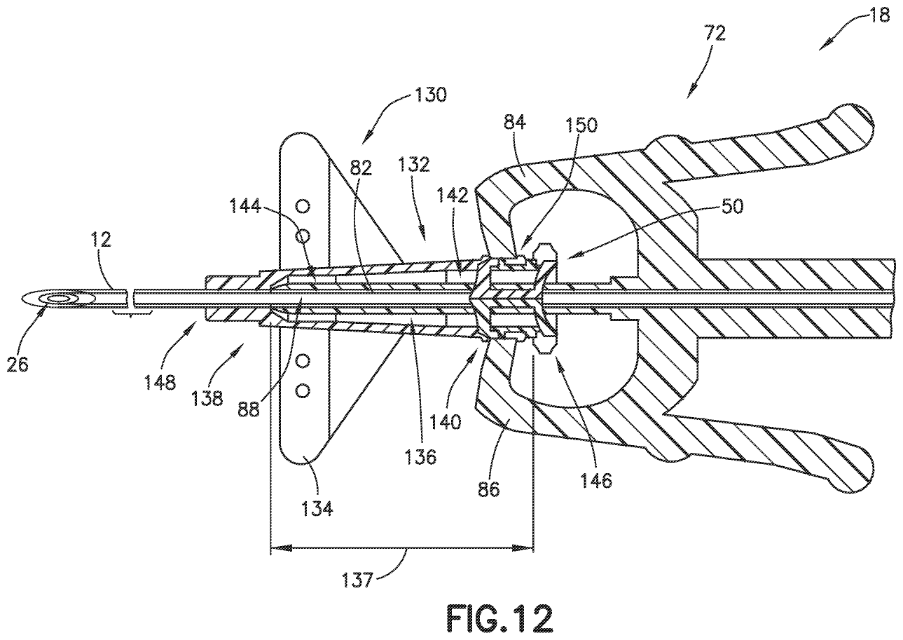

Referring now to FIGS. 11 and 12, a catheter hub 130 directly coupleable to an instrument delivery device 18 of a catheter system 10 is shown in accordance with another aspect or embodiment. The catheter hub 130 is constructed so as to be similar to the catheter hub 14 shown in FIGS. 1-10, in that the catheter hub 130 may include a main hub body 132 and stabilizing wings 134 extending out from the main hub body 132. However, catheter hub 130 is configured as a single-ported catheter hub that does not include a side port thereon.

The main hub body 132 of catheter hub 130 defines a cavity 136 therein that extends between distal and proximal ends 138, 140 of the catheter hub 130. According to aspects or embodiments of the disclosure, a proximal end portion 142 of the cavity 136 is configured as a tapered female luer cavity, while a distal end portion 144 of the cavity 136 is configured as a generally cylindrically shaped cavity that has a constant diameter along at least a majority of a length 37 thereof. A hub end port 146 is provided at the proximal end 140 of the main hub body 132 that provides access into the cavity 136. An opening 148 is also formed at the distal end 138 of the main hub body 132 that extends into the cavity 136, with the opening 148 also receiving the proximal end 22 of the catheter 12 therein.

In accordance with some aspects and embodiments, the hub end port 146 at the proximal end 140 of main hub body 132 defines the tapered proximal end portion 142 of the cavity 136 and is configured to receive a split-septum connector 50 therein. As shown in FIG. 12, the hub end port 146 may be configured so as to accommodate an entirety of the split-septum connector 50 therein, such that the split-septum connector 50 is internal to the catheter hub 130. With the split-septum connector 50 positioned internal to the catheter hub 130, the hub end port 146 may include a groove 150 formed on an outer surface thereof that provides a location for connection of a blood draw device 18 (or other instrument delivery device) thereto—with arms 84, 86 on the connector 72 of blood draw device 18 engaging the catheter bub within groove 150.

In engaging the blood draw device 18 to the catheter hub 130, the configuration of the distal end portion 144 of the cavity 136—with a cylindrical profile and a length 37 designed to accommodate the full length of the blunt cannula 82—allows the blunt cannula 82 to be received and accurately guided through the cavity 136 without obstruction when the connector 72 is engaged with the catheter hub 130. With the blunt cannula 82 extending entirely through the cavity 136, the lumen 88 of the connector 72 may be positioned directly adjacent the lumen 26 of catheter 12 (or adjacent the lumen 48 of a wedge 42, if included). The construction of catheter hub 130 and the mating thereof with blood draw device 18 thus beneficially facilitates advancement of the secondary catheter 74 from the first position to the second position, with the secondary catheter 74 advancing through the lumen 88 of connector 72 and into the lumen 26 of catheter 12 in a straight line-without any opportunity for the secondary catheter 74 (FIGS. 4 and 5, for example) to deflect or otherwise become trapped/caught within the cavity 136 of the catheter hub 130.

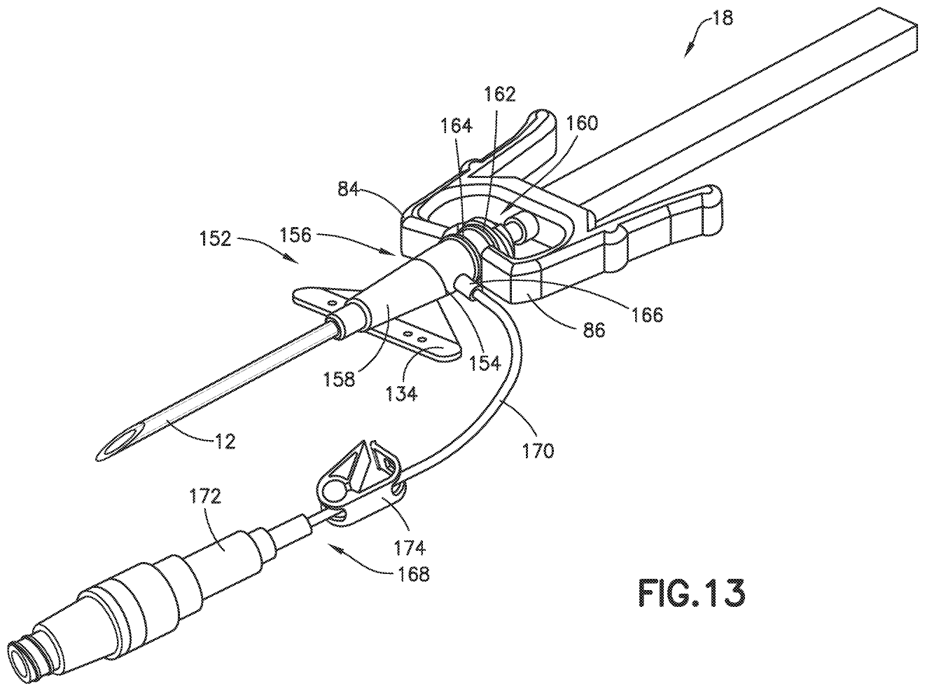

Referring now to FIG. 13, a catheter hub 152 directly coupleable to an instrument delivery device 18 of a catheter system 10 is shown in accordance with another aspect or embodiment. The catheter hub 152 is similar to the catheter hub 130 of FIGS. 11 and 12 in that catheter hub 152 is configured as a single-ported catheter hub that does not include a side port thereon; however, catheter hub 152 is constructed such that a hub end port 154 at the proximal end 156 of main hub body 158 is configured to receive only a portion of a proximal coupler 160 therein (rather than an entirety of a split-septum connector 50, as in FIGS. 11 and 12). As previously described, the proximal coupler 160 may be configured as a NFC that includes a split septum therein that serves as an access control feature whereby a peripheral device (e.g., a blood draw device or a vascular access probe for in-vein digital measurement of patient data such as temperature, pH, lactate, and/or other blood-based measurements) can be physically and fluidically coupled to the catheter hub 152. As can be seen in FIG. 13, the proximal coupler 160 includes a pair of connection rings 162, 164 formed about a perimeter thereof, with a first ring 162 and a second ring 164 spaced laterally apart, so as to provide for engagement of the instrument delivery device 18 (i.e., arms 84, 86) to the catheter hub 152. Additionally, proximal coupler 160 includes a side port 166 formed integrally thereon that is distal from the split-septum valve therein. The side port 166 can be disposed substantially perpendicular to a lumen formed in/through the coupler 160. An extension set 168 may be integrated with side port 166 that can be used to deliver fluid, remove fluid, flush fluid, and/or the like. Extension tubing 170 may extend out from side port 166, with a proximal access port 172 coupled to a proximal end portion of the extension tubing 170 to control access to the extension set 168. A clamp 174 may also be provided on the extension tubing 170, with the clamp 174 configured to selectively restrict flow through the extension tubing 170.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A catheter system comprising:
a catheter having a catheter distal end and a catheter proximal end, the catheter defining a catheter lumen;
a catheter hub coupled to the catheter proximal end, the catheter hub comprising:
a hub body having a hub distal end and a hub proximal end, the hub body having a cavity formed therein extending between the hub distal end and the hub proximal end; and
an end port positioned at the hub proximal end and providing an opening into the cavity; and
a split-septum connector positioned at least partially within the end port, the split-septum connector configured to seal the cavity when in a closed configuration and provide a fluid path to the catheter lumen when in an open configuration.

2. The catheter system of claim 1, wherein the catheter hub further comprises a side port extending out from the hub body between the hub distal end and the hub proximal end, the side port defining a side port lumen in fluid communication with the catheter lumen.

3. The catheter system of claim 2, wherein the side port extends out from the hub body at an angle relative to the catheter lumen of between 15-165 degrees.

4. The catheter system of claim 2, wherein the side port comprises a female luer connection.

5. The catheter system of claim 2, further comprising a needle-free access connector coupled to the side port.

6. The catheter system of claim 1, wherein the cavity comprises a cylindrical cavity having a constant diameter along at least a majority of a length thereof between the hub distal end and the hub proximal end.

7. The catheter system of claim 1, wherein the catheter system further comprises a blood draw device, the blood draw device coupled to the end port and/or the split-septum connector.

8. The catheter system of claim 7, wherein the blood draw device comprises:
a secondary catheter;
an introducer having a proximal end and a distal end, and a top surface and a bottom surface, that define an inner volume configured to movably receive the secondary catheter;
an actuator movably coupled to the introducer, the actuator configured to move relative to the introducer to move a distal end of the secondary catheter from a position within the introducer to a position outside the introducer housing and past the catheter distal end of the catheter; and
a connector positioned at the distal end of the introducer that is coupled to the end port and/or the split-septum connector, the connector comprising a blunt cannula and a pair of locking arms.

9. The catheter system of claim 8, wherein the split-septum connector comprises a first ring and a second ring spaced apart laterally from the first ring, and wherein the pair of locking arms engages the split-septum connector by snapping-in between the first ring and the second ring.

10. The catheter system of claim 8, wherein when the blood draw device is coupled to the end port and/or the split-septum connector, the blunt cannula penetrates through the split-septum connector and into the cavity, with each of the blunt cannula and the cavity having a length such that the blunt cannula extends approximately to a distal end of the cavity.

11. The catheter system of claim 10, wherein extension of the blunt cannula to approximately the distal end of the cavity places a lumen of the blunt cannula adjacent the catheter lumen.

12. The catheter system of claim 11, wherein the catheter hub further comprises a wedge positioned within the cavity adjacent the hub distal end, the wedge coupled to the catheter proximal end to retain the catheter to the catheter hub, and wherein the wedge includes a wedge lumen formed therein, and extension of the blunt cannula to approximately the distal end of the cavity directly aligns the lumen of the blunt cannula with the wedge lumen.

13. The catheter system of claim 8, wherein the split-septum connector is positioned entirely within the end port, and wherein the pair of locking arms engages a groove formed on an outer surface of the end port.

14. The catheter system of claim 1, further comprising a needle introducer coupled to the split-septum connector, with a needle extending through the catheter hub and through the catheter lumen, and wherein the split-septum connector is configured to have an initial configuration where a first portion of the split-septum connector is separated from a second portion of the split-septum connector, such that the needle passes through the split-septum connector without making contact therewith.

15. The catheter system of claim 1, wherein the catheter hub comprises an open peripheral intravenous catheter (PIVC) catheter hub.

16. The catheter system of claim 1, wherein the split-septum connector comprises a side port formed thereon, with an integrated extension tube extending out from the side port.

17. A catheter system comprising:
a catheter having a catheter distal end and a catheter proximal end, the catheter defining a catheter lumen;
a catheter hub coupled to the catheter proximal end, the catheter hub comprising:
a hub body having a hub distal end and a hub proximal end, the hub body having a cavity formed therein extending between the hub distal end and the hub proximal end; and
an end port positioned at the hub proximal end and providing an opening into the cavity; and
a fluid cut-off positioned at or adjacent the end port, the fluid cut-off configured to seal the cavity when in a closed configuration and provide a fluid path to the catheter lumen when in an open configuration.

18. The catheter system of claim 17, wherein the fluid cut-off comprises:
a stop cock valve within the cavity adjacent the end port; and
an actuator positioned on an outer surface of the hub body and operable to open and close the stop cock valve.

19. The catheter system of claim 17, further comprising a split-septum connector within the cavity and proximal from the stop cock valve.

20. The catheter system of claim 19, further comprising a deformable tubing coupled to the end port and extending our proximally therefrom, and wherein the fluid cut-off comprises a slide clamp positioned on the deformable tubing and actuatable between an open position and a closed position to selectively allow fluid flow through the deformable tubing.

21. The catheter system of claim 19, wherein the catheter system further comprises a blood draw device coupleable to the proximal coupler of the connector, the blood draw device configured to advance a secondary catheter past the catheter distal end of the catheter, the blood draw device comprising a connector that is coupled to the end port, the connector comprising a blunt cannula and a pair of locking arms; and
wherein, when the blood draw device is coupled to the end port and when the fluid cut-off is in the open configuration, the blunt cannula penetrates into the cavity, with the blunt cannula and the cavity each having a length such that the blunt cannula extends approximately to a distal end of the cavity.
